(19) United States
(12) Patent Application Publication
(10) Pub. No.: US 2023/0265478 A1
(43) Pub. Date: Aug. 24, 2023

(54) METHODS OF INCREASING RECOMBINANT PROTEIN YIELDS

(71) Applicant: BIOTALYS NV, Sint-Denijs-Westrem (BE)

(72) Inventors: Lucia Nancy Coconi Linares, Sint-Denijs-Westrem (BE); Jolien Van Poucke, Sint-Denijs-Westrem (BE); Charlotte Verlinde, Sint-Denijs-Westrem (BE)

(21) Appl. No.: 18/007,340

(22) PCT Filed: Aug. 2, 2021
(86) PCT No.: PCT/EP2021/071596

§ 371 Date: Jan. 30, 2023

(30) Foreign Application Priority Data
Jul. 31, 2020 (EP) 20188921.9
Jul. 31, 2020 (EP) 20188933.4
Dec. 30, 2020 (EP) 20217907.3

(51) Int. Cl. C12P 21/02; C07K 16/00

(52) U.S. Cl. CPC C12P 21/02; C07K 16/00; C07K 2317/14; C07K 2317/569

(57) ABSTRACT

The present invention relates to methods for increasing yield of compounds of interest produced by microbial cells, in particular recombination proteins produced by microbial cells. The present invention also relates to the use of peptone as a yield increasing agent in a method of production of a compound of interest. The present invention provides the compounds of interest, such as recombinant proteins, obtained by the method of the invention.

### FIELD OF THE INVENTION

The present invention relates to methods for increasing yield of compounds of interest produced by microbial cells, in particular recombination proteins produced by microbial cells. The present invention also relates to the use of peptone as a yield increasing agent in a method of production of a compound of interest. The present invention provides the compounds of interest, such as recombinant proteins, obtained by the method of the invention.

Other aspects, embodiments, advantages and applications of the invention will become clear from the further description herein.

### BACKGROUND

The efficient and cost-effective production of recombinant proteins is very important in the field of pharmacology but even more so in the field of agriculture where greater amounts of active protein may be required at low cost price. This puts high demands on the production process and development of biological products.

Different species of filamentous fungi have historically been used in fermentations and were selected by centuries of use. In more recent times, filamentous fungi are being used for their properties to produce extracellular plant biomass-degrading enzymes. This interesting aspect was mainly exploited with the production of biofuels as a goal. The key producers of extracellular (hemi)-cellulases are Aspergillus, Trichoderma, Penicillium and Neurospora species and over the past decades these strains have been improved using random mutagenesis, selection and genetic engineering with some species and strains now reported to produce up to 100 g/l of extra-cellular (hemi)cellulases (Cherry J R, Fidantsef A L, Opin. Biotechnol. 14(4), 438-443). Such protein production levels have spurred researchers to try and utilize filamentous fungi for the production of recombinant proteins by using strong endogenous promoters, signal peptides, and carrier (hemi)cellulolytic genes fused to the target genes. Very often however, these attempts did not produce the desired or hoped for expression levels of recombinant proteins. For example, during the production of a biological product, such as conventional monoclonal antibodies, unsatisfactory yields were reported ranging from 0.15 g/l in T. reesei to 0.9 g/l in A. niger. Such low amounts of biological product are insufficient for profitable production of proteins in industrial biotechnology, pharmacological and agricultural applications (Nyyssonen et al, 1993, Biotechnology. 11; Ward et al 2004, Appl. Environ. Microbiol. 70).

Many efforts have been undertaken to increase expression levels from filamentous fungi, such as searching for new promoters, deleting regulators such as catabolite repression modulators, introduction of chaperones, and so forth (Nevalainen, 2004, Handbook of fungal biotechnology). But despite all these previous and ongoing efforts, no substantial progress has yet been reported in the yields of recombinant protein production in fungal hosts (Nevalainen et al 2014, Front. Microbiol. 5:75).

A key reason might be the rapid degradation of secreted (heterologous) recombinant proteins by the presence of extracellular proteases. Indeed, filamentous fungi are well-known for secreting a wide variety and large amounts of proteases into the environment. Proteins that are unstable or sensitive to protease degradation will therefore quickly be degraded. This results in very low protein yields or even the total absence of protein recovery after fermentation due to large quantities of these proteases in the fermentation broth. Additionally, the presence of proteases in a protein formulation, be it even at low quantities, may greatly impact the shelf life of protein products. Therefore, many attempts have been undertaken to reduce the protease activity and hence stability of recombinant proteins in the culture media. A tested approach is the deletion of each individual protease as identified by homology searches and certain discernible patterns shared by commonly known proteases. WO2013102674, WO2015004241 and WO2007045248 describe Trichoderma mutants with a plurality of individually modified protease genes in an attempt to reduce degradation of a recombinantly produced biological products. In addition to the laborious and time-consuming procedure to modify each protease sequentially, it also comes with the drawback that it is limited to those proteases that have been identified by experimental or bio-informatic analysis. It is likely that many proteases remain unidentified. And even if all proteases are identified, the deletion of all of them would ideally be necessary.

Alternatively, protease regulators are modified. WO2017025586 reports the modification of T. reesei genes that share characteristics of regulators of transcription. This identification was also based on the proximity of those regulators to protease genes or clusters of proteases. The inactivation of 3 putative regulators and the deletion of 8 individual proteases led to decreases in protease production and increased yields in interferon production of 3.7-fold compared to a parent strain. WO2017025586 does not test production of other biologicals such as traditional monoclonal antibodies.

WO2016132021 describes the inactivation of a newly discovered regulator, peal, and reports reduced protease activity to a level of 25-50% compared to that of wild type levels in Trichoderma reesei and a 40-fold reduction in protease levels when peal was inactivated in Fusarium oxysporum. No increases in protein production yields were reported, however.

Qian et al. (2019) report the deletion of a regulator, Aare1, in the Trichoderma reesei strain QM9414. Whilst reporting decreased expression of two proteases Aapw1 (Uniport ID: GOR8T0) and Aapw2 (Uniprot ID: GOR9K1), no increased production or improved stability of a compound of interest was reported. Unfortunately, the Are1 deletion in QM9414 drastically reduced expression of the major cellulase proteins Cbh1 and Cbh2 in the presence of ammonium sulphate and peptone, reducing the industrial applicability of this modified Trichoderma strain since cbh1 and cbh2 promoters are very often used to drive expression of a heterologous protein to high levels.

Although WO2013102674, WO2015004241 and WO2007045248 report the specific inactivation of proteases and WO2017025586, WO2016132021 and Qian et al (2019) report the inactivation of specific genetic regulators, with both approaches leading to a decreased protease content, reported results in the reduction of recombinant protein degradation remain highly specific for the chosen protease, highly variable or even absent. Significant increases in protein yields from large scale fermentations remain to be reported.

There remains a need in the art for still further improved host cells, for example filamentous fungal cells, such as Trichoderma fungus cells, that can stably produce heterologous proteins, such as immunoglobulins, preferably at high levels of expression.

### SUMMARY OF THE INVENTION

The present invention provides methods for the production of compounds of interest, in particular recombinant proteins.

In a first aspect of the invention there is provided a method for the production of a compound of interest comprising:

providing a microbial host cell comprising at least one polynucleotide coding for a compound of interest;

culturing said microbial host cell under conditions conducive to the expression of the compound of interest, wherein the microbial host is cultured in the presence of peptone.

In a second aspect of the invention, there is provided the use of peptone as a yield increasing agent in a method of production of a compound of interest, wherein the method comprises:

providing a microbial host cell capable of expressing the compound of interest;

culturing said microbial host cell under conditions conducive to the expression of a compound of interest, wherein the microbial host is cultured in the presence of peptone.

In a third aspect of the invention, there is provided the use of a microbial host cell for the production of a compound of interest, wherein the microbial host comprises at least one polynucleotide coding for the compound of interest, and wherein the method comprises culturing the microbial host cell under conditions conducive to the expression of the compound of interest, wherein the microbial host is cultured in the presence of peptone.

In a fourth aspect of the invention, there is provided a kit of parts, wherein the kit comprises peptone plus various additional components such as a microbial host cells, a vector encoding a compound of interest, and/or a vector for homologous recombination of a microbial cell, for example for effecting a full or partial deletion of at least one polypeptide encoded by the genome of the microbial cell, where the at least one polypeptide is a regulator of transcription that controls the expression of one or more proteases.

### BRIEF DESCRIPTION OF THE FIGURES

FIG. 1: SDS-PAGE analysis of extracellular proteins of Trichoderma reesei after 6 days post-lactose induction in either Vogel's medium with ammonium or peptone as a nitrogen source. CTL shows the pure VHH-1 as a reference.

FIG. 2: pNP-cellobiohydrolase assays of Trichoderma reesei until 11 days of fermentation.

FIG. 3: Protease concentration in supernatants of controlled batch fermentations of T. reesei RL-P37 grown in minimal medium with two different nitrogen sources, peptone, and ammonium.

FIG. 4: Comparison of protein abundance of CBHI and CBHII produced by RL-P37 strain after 6 days of fermentation on minimal medium with and without peptone. The bars show the label free quantification (LFQ) intensities associated with the cellulase abundance.

FIG. 5: Comparison of VHH-1 protein production by Trichoderma reesei RL-P37 on minimal media supplemented with either peptone (P), ammonium (A), or a combination of peptone and ammonium (P+A).

### BRIEF DESCRIPTION OF THE SEQUENCE LISTING

SEQ ID NOs: 1 to 5 are the sequence of VHH-1, where SEQ ID NO: 1 is the full length sequence of VHH-1, SEQ ID NO: 2 is the full length sequence of VHH-1 but in which the first residue is changed to a Q residue, SEQ ID NO: 3 is the CDR1 of VHH-1, SEQ ID NO: 4 is the CDR2 of VHH-1 and SEQ ID NO: 5 is the CDR3 of VHH-1.

SEQ ID NOs: 6 to 10 are the sequences of VHH-2, where SEQ ID NO: 6 is the full length sequence of VHH-1, SEQ ID NO: 7 is the full length sequence of VHH-2 but in which the first residue is changed to a Q residue, SEQ ID NO: 8 is the CDR1 of VHH-2, SEQ ID NO: 9 is the CDR2 of VHH-2 and SEQ ID NO: 10 is the CDR3 of VHH-2.

SEQ ID NOs: 11 to 15 are the sequences of VHH-3, where SEQ ID NO: 11 is the full length sequence of VHH-1, SEQ ID NO: 12 is the full length sequence of VHH-3 but in which the first residue is changed to a Q residue, SEQ ID NO: 13 is the CDR1 of VHH-3, SEQ ID NO: 14 is the CDR2 of VHH-3 and SEQ ID NO: 15 is the CDR3 of VHH-3.

### DETAILED DESCRIPTION OF THE INVENTION

Reference to any prior art in this specification is not, and should not be taken as, an acknowledgment or any form of suggestion that this prior art forms part of the common general knowledge in any country.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs.

The present invention will be described with respect to particular embodiments but the invention is not limited thereto but only by the claims. Any reference signs in the claims shall not be construed as limiting the scope.

Where the term “comprising” is used in the present description and claims, it does not exclude other elements or steps.

Where an indefinite or definite article is used when referring to a singular noun e.g. “a” or “an”, “the”, this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The following invention relates to methods on increasing the yield of compounds of interest, in particular recombinant proteins, expressed by microbial cells.

For example, it has been surprisingly found that when the microbial host cell capable of expressing a compound of interest is cultured in the presence of peptone, an improved yield of said compound is obtained if compared to a method in which the host cell is cultured and measured under the same or substantially the same conditions, but without peptone.

The methods according to this invention can be useful for the industrial production of compounds of interest such as polypeptides. The polypeptides may be useful in the preparation of agrochemical or pharmaceutical compositions.

Microbial Host Cells Used in the Invention and Methods for Making them

The present invention uses microbial cells, specifically microbial host cells. Within the context of the present invention “measured under the same conditions” or “measured under substantially the same conditions” means that the microbial host cell which is capable of producing a compound of interest is cultured under the same conditions with the exception of the presence or absence of peptone in the cell culture medium, preferably by using the same assay and/or methodology, more preferably within the same experiment, to determine the effect of the presence or absence of peptone on the yield of the compound of interest. The same conditions refers to the culture conditions used to culture the microbial host cell, with the exception of the presence or absence of peptone. In some contexts, the cell culture medium may comprise an additional component to compensate for the absence of peptone. This is because the peptone may be acting as a source of nitrogen for the microbial cell. If peptone is absent from the cell culture medium, an alternative nitrogen source may be required. For example, in some embodiments, “measured under the same conditions” or “measured under substantially the same conditions” means the microbial host cell which is capable of producing a compound of interest is cultured under the same conditions with the exception of the exchange of peptone for a different component. More specifically, “measured under the same conditions” or “measured under substantially the same conditions” may mean the microbial host cell which is capable of producing a compound of interest is cultured under the same conditions with the exception of the exchange of peptone for a different component that acts as a source of nitrogen. Therefore, “measured under the same conditions” or “measured under substantially the same conditions” may be described as culturing the microbial cell in the presence or absence of peptone, wherein when peptone is absent from the cell culture medium, it is optionally replaced with an alternative nitrogen source. Alternative nitrogen sources include, for example, ammonium. The skilled person will be aware of the nutritional requirements of the microbial cells being cultured, and thus will know if, when peptone is absent from the cell culture medium, whether it needs to be replaced with an alternative source of nitrogen to ensure survival of the microbial host cell.

In some embodiments, the method for measuring the yield of the compound of interest comprises providing a microbial cell, culturing the microbial cell in a cell culture medium comprising peptone, spiking the culture broth with a test compound of interest (i.e. adding a quantity of test compound of interest to the cell culture medium) and measuring the extent of the degradation of the compound of interest in the culture broth overtime. The method may further comprise providing a microbial cell, culturing the microbial cell in a cell culture medium in the absence of peptone (optionally wherein the peptone is replaced with an alternative component that provides a source of nitrogen), spiking the culture broth with a test compound of interest (i.e. adding a quantity of test compound of interest to the cell culture medium) and measuring the extent of the degradation of the compound of interest in the culture broth over time. The method may then comprise comparing the degradation of the compound of interest over time when the microbial cell is cultured in the presence of peptone with the degradation of the compound of interest over time when then the microbial cell is cultured in the absence of peptone. In methods of this type, the microbial cell does not need to comprise a polynucleotide encoding for the compound of interest (and hence may be a parental microbial cell, which does not comprise said polynucleotide).

In some embodiments, the method for measuring protease activity comprises culturing the microbial host cell comprising at least one polynucleotide coding for a compound of interest in a cell culture medium comprising peptone under conditions to cause production of the compound of interest by the microbial host cell, obtaining one or more samples of the liquid cell culture medium at periodic intervals and measuring the concentration of the compound of interest in each sample to determine the protease activity of the microbial host cell. The method may further comprise carrying out the same method on a microbial host cell comprising at least one polynucleotide coding for a compound of interest, but using a cell culture medium that does not comprise peptone (optionally wherein the peptone is replaced with an alternative component that provides a source of nitrogen), and comparing the concentration of the compound of interest in the cell culture medium without peptone with the concentration of the compound of interest in the cell culture medium with peptone to quantify a change in compound yield caused by the presence or absence of peptone.

In some embodiments obtaining a sample of the culture broth can include the step of removing the microbial host cell before obtaining a sample, or a sample of the culture broth can contain both the culture broth as the microbial host cell, or the microbial host cell can be lysed prior to taking a sample of the culture broth.

As the production of the compound of interest over a period of time, when making comparisons in the yield between microbial host cells cultured either in the presence or absence of peptone, the skilled person will be aware the comparison may be made using yield measurement determined after the same culture time (i.e. after the microbial host cells used in to the two comparative experiments have been cultured for the same length of time). In addition, or as an alternative, the skilled person will be aware that the comparisons may be made using yield measurements from cultures that contain a similar amount of the microbial host cells. The skilled person will be aware that the comparison may be made using yield measurements starting from samples containing similar amounts of the microbial host cell (i.e. by making appropriate dilutions or concentrating samples before measurements).

A “parent microbial cell” or “parental microbial cell” is defined as a microbial cell that does not comprise the at least one polynucleotide coding for a compound of interest (and hence may be referred to as an unmodified microbial cell). The parent microbial cell will generally be genetically identical to the microbial host cell, with the exception of the presence in the microbial host cell of at least one polynucleotide coding for a compound of. The parent microbial cell may therefore be considered a wild-type cell (and is referred to herein as such), since the host has not been modified to include the at least one polynucleotide coding for a compound of interest.

A “microbial host cell” is herewith defined as a microbial host cell derived from a parent cell and which has been modified to incorporate the at least one polynucleotide coding for a compound of interest.

A microbial host cell is defined here as a single cellular organism used during a fermentation process or during cell culture to produce a compound of interest. Preferably, a microbial host cell is selected from the kingdom Fungi. In particular, the fungus may be a filamentous fungus.

The fungi may preferably be from the division Ascomycota, subdivision Pezizomycotina. In some embodiments, the fungi may preferably from the Class Sordariomycetes, optionally the Subclass Hypocreomycetidae. In some embodiments, the fungi may be from an Order selected from the group consisting of Hypocreales, Microascales, Eurotiales, Onygenales and Sordariales. In some embodiments, the fungi may be from a Family selected from the group consisting of Hypocreaceae, Nectriaceae, Clavicipitaceae and Microascaceae. In some more specific embodiments, the fungus may be from a Genus selected from the group consisting of Trichoderma (anamorph of Hypocrea), Myceliophthora, Fusarium, Gibberella, Nectria, Stachybotrys, Claviceps, Metarhizium, Villosiclava, Ophiocordyceps, Cephalosporium, Rasamsonia, Neurospora, and Scedosporium. In some further and more specific embodiments, the fungi may be selected from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Neurospora crassa, Rasamsonia emersonii, Acremonium (Cephalosporium) chrysogenum, Scedosporium apiospermum, Aspergillus niger, A. awamori, A. oryzae, Chrysosporium lucknowense, Myceliophthora thermophila, Myceliophthora heterothallica, Humicola insolens, and Humicola grisea, most preferably Trichoderma reesei. If the host cell is a Trichoderma reesei cell, it may be selected from the following group of Trichoderma reesei strains obtainable from public collections: QM6a, ATCC13631; RutC-30, ATCC56765; QM9414, ATCC26921, RL-P37 and derivatives thereof. If the host cell is a Myceliophthora heterothallica, it may be selected from the following group of Myceliophthora heterothallica or Thermothelomyces thermophilus strains: CBS 131.65, CBS 203.75, CBS 202.75, CBS 375.69, CBS 663.74 and derivatives thereof. If the host cell is a Myceliophthora thermophila it may be selected from the following group of Myceliophthora thermophila strains ATCC42464, ATCC26915, ATCC48104, ATCC34628, Thermothelomyces heterothallica C1, Thermothelomyces thermophilus M77 and derivatives thereof. If the host cell is an Aspergillus nidulans it may be selected from the following group of Aspergillus nidulans strains: FGSC A4 (Glasgow wild-type), GR5 (FGSC A773), TN02A3 (FGSC A1149), TN02A25, (FGSC A1147), ATCC 38163, ATCC 10074 and derivatives thereof.

With a “compound of interest” it is meant any recombinant protein such as an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH.

In some embodiments, the compound of interest is a therapeutic protein, biosimilar, multi-domain protein, peptide hormone, antimicrobial peptide, peptide, carbohydrate binding module, enzyme, cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase or xylanase.

In some embodiments, the compound of interest is a VHH. In more specific embodiments, the VHH may be a VHH bind a specific lipid fraction of the cell membrane of a fungal spore. Such VHHs may exhibit fungicidal activity through retardation of growth and/or lysis and explosion of spores, thus preventing mycelium formation. The VHH may therefore have fungicidal or fungistatic activity.

In some embodiments, the VHH may be a VHH that is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:

fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The invention also provides a polypeptide, wherein said at least one polypeptide is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:

fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The VHHs are generally capable of binding to a fungus. The VHH thereby causes retardation of growth of a spore of the said fungus and/or lysis of a spore of the said fungus. That is to say, binding of the VHH to a fungus results in retardation of growth of a spore of the said fungus and/or lysis of a spore of the said fungus.

The VHHs may (specifically) bind to a membrane of a fungus or a component of a membrane of a fungus. In some embodiments, the VHHs do not (specifically) bind to a cell wall or a component of a cell wall of a fungus. For example, in some embodiments, the VHHs do not (specifically) bind to a glucosylceramide of a fungus.

The VHHs may be capable of (specifically) binding to a lipid-containing fraction of the plasma membrane of a fungus, such as for example a lipid-containing fraction of Botrytis cinerea or other fungus. Said lipid-containing fraction (of Botrytis cinerea or otherwise) may be obtainable by chromatography. The chromatography may be performed on a crude lipid extract (also referred to herein as a total lipid extract, or TLE) obtained from fungal hyphae and/or conidia. The chromatography may be, for example, thin-layer chromatography or normal-phase flash chromatography. The chromatography (for example thin-layer chromatography) may be performed on a substrate, for example a glass plate coated with silica gel. The chromatography may be performed using a chloroform/methanol mixture (for example 85/15% v/v) as the eluent.

For example, said lipid-containing fraction may be obtainable by a method comprising:

fractionating hyphae and/or conidia of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

In a more specific embodiment, the lipid-containing fraction may be obtainable by a method comprising:

fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (Rf) higher than the ceramide fraction and lower than the non-polar phospholipids fraction.

Alternatively, the fraction may be obtained using normal-phase flash chromatography. In such a method, the method may comprise:

fractionating hyphae and/or conidia of a fungus (for example Botrytis cinerea or other fungus) by total lipid extract normal-phase flash chromatography, and selecting the fraction with a Retention Factor (Rf) higher than the ceramide fraction and lower than the non-polar phospholipids fraction.

In a more specific embodiment, the lipid-containing fraction may be obtainable by a method comprising:

fractionating hyphae and/or conidia of a fungus (for example Botrytis cinerea or other fungus) by total lipid extract normal-phase flash chromatography comprising dissolving the TLE in dichloromethane (CH2Cl2) and MeOH and using CH2Cl2/MeOH (for example 85/15%, v/v) as the eluent, followed by filtration of the fractions through a filter.

In a more specific embodiment, the lipid-containing fraction may be obtainable by a method comprising:

fractionating hyphae and/or conidia of a fungus (for example Botrytis cinerea or other fungus) by total lipid extract normal-phase flash chromatography comprising dissolving the TLE in dichloromethane (CH2Cl2) and MeOH loading the TLE on to a phase flash cartridge (for example a flash cartridge with 15 μm particles), running the column with CH2Cl2/MeOH (85/15%, v/v) as the eluent, and filtering the fractions through a filter (for example a 0.45 μm syringe filter with a nylon membrane) and drying the fractions.

The fractions from the chromatography may be processed prior to testing of binding of the VHH to the fraction or of interaction with the fraction. For example, liposomes comprising the fractions may be prepared. Such a method may comprise the use of thin-film hydration. For example, in such a method, liposomes may be prepared using thin-film hydration with the addition of 1,6-diphenyl-1,3,5-hexatriene (DPH). Binding and/or disruption of the membranes by binding of the VHH may be measured by a change in fluorescence before and after polypeptide binding (or by reference to a suitable control).

Accordingly, in some embodiments, the VHHs may (specifically) bind to a lipid-containing chromatographic fraction of the plasma membrane of a fungus, optionally wherein the lipid-containing chromatographic fraction is prepared into liposomes prior to testing the binding of the polypeptide thereto.

Binding of the VHH to a lipid-containing fraction of a fungus may be confirmed by any suitable method, for example bio-layer interferometry. Specific interactions with the lipid-containing fractions may be tested. For example, it may be determined if the polypeptide is able to disrupt the lipid fraction when the fraction is prepared into liposomes, for example using thin-film hydration.

In methods involving chromatography, an extraction step may be performed prior to the step of chromatography. For example, fungal hyphae and/or conidia may be subjected to an extraction step to provide a crude lipid extract or total lipid extract on which the chromatography is performed. For example, in some embodiments, fungal hyphae and/or conidia (for example fungal hyphae and/or conidia of Fusarium oxysporum or Botrytis cinerea) may be extracted at room temperature, for example using chloroform:methanol at 2:1 and 1:2 (v/v) ratios. Extracts so prepared may be combined and dried to provide a crude lipid extract or TLE.

Accordingly, in some embodiments, the VHH may be capable of (specifically) binding to a lipid-containing fraction of the plasma membrane of a fungus (such as Fusarium oxysporum or Botrytis cinerea), wherein the lipid-containing fraction of the plasma membrane of the fungus is obtained or obtainable by chromatography. The chromatography may be normal-phase flash chromatography or thin-layer chromatography. Binding of the VHH to the lipid to the lipid-containing fraction may be determined according to bio-layer interferometry. In some embodiments, the chromatography step may be performed on a crude lipid fraction obtained or obtainable by a method comprising extracting lipids from fungal hyphae and/or conidia from a fungal sample. The extraction step may use chloroform:methanol at 2:1 and 1:2 (v/v) ratios to provide two extracts, and then combining the extracts.

In methods relating to thin-layer chromatography, the chromatography may comprise the steps of:

fractionating hyphae of the fungus by total lipid extract thin-layer chromatography and selecting the fraction with a Retention Factor (Rf) higher than the ceramide fraction and lower than the non-polar phospholipids fraction.

In some methods relating to thin-layer chromatography, the chromatography may comprise the steps of:

fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (Rf) higher than the ceramide fraction and lower than the non-polar phospholipids fraction.

In methods relating to normal-phase flash chromatography, the chromatography may comprise the steps of:

fractionating hyphae and/or conidia of a fungus (for example Botrytis cinerea or other fungus) by total lipid extract normal-phase flash chromatography, and selecting the fraction with a Retention Factor (Rf) higher than the ceramide fraction and lower than the non-polar phospholipids fraction.

In some methods relating to normal-phase flash chromatography, the chromatography may comprise the steps of:

fractionating hyphae and/or conidia of a fungus (for example Botrytis cinerea or other fungus) by total lipid extract normal-phase flash chromatography comprising dissolving the TLE in dichloromethane (CH2Cl2) and MeOH and using CH2Cl2/MeOH (for example 85/15%, v/v) as the eluent, followed by filtration of the fractions through a filter.

In some methods relating to normal-phase flash chromatography, the chromatography may comprise the steps of:

fractionating hyphae and/or conidia of a fungus (for example Botrytis cinerea or other fungus) by total lipid extract normal-phase flash chromatography comprising dissolving the TLE in dichloromethane (CH2Cl2) and MeOH loading the TLE on to a phase flash cartridge (for example a flash cartridge with 15 μm particles), running the column with CH2Cl2/MeOH (85/15%, v/v) as the eluent, and filtering the fractions through a filter (for example a 0.45 μm syringe filter with a nylon membrane) and drying the fractions.

In some embodiments, the compound of interest is VHH-1, VHH-2 or VHH-3. For example, in some embodiments, the compound of interest is a VHH comprising or consisting of a sequence selected from the group consisting of SEQ ID NOs: 1, 2, 6, 7, 11 and 12.

In some embodiments, the compound of interest is a VHH comprising:
(a) a CDR1 comprising or consisting of a sequence selected from the group consisting of SEQ ID NOs 3, 8 and 13;
(b) a CDR2 comprising or consisting of a sequence selected from the group consisting of SEQ ID NOs: 4, 9 and 14; and
(c) a CDR3 comprising or consisting of a sequence selected from the group consisting of SEQ ID NOs: 5, 10 and 15.

In some embodiments, the compound of interest is a VHH comprising:
(a) a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5;
(b) a CDR1 comprising or consisting of the sequence of SEQ ID NO: 8, a CDR2 comprising or consisting of the sequence of SEQ ID NO: 9 and a CDR3 comprising or consisting of the sequence of SEQ ID NO: 10 or
(c) a CDR1 comprising or consisting of the sequence of SEQ ID NO: 13, a CDR2 comprising or consisting of the sequence of SEQ ID NO: 14 and a CDR3 comprising or consisting of the sequence of SEQ ID NO: 15.

In some embodiments, the compound of interest is a VHH comprising a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5.

In some embodiments, the compound of interest is a VHH comprising SEQ ID NO: 1.

In some embodiments, the compound of interest is a VHH comprising SEQ ID NO: 2.

In some embodiments, the compound is a VHH disclosed in WO2014/177595 or WO2014/191146, the entire contents of which are incorporated herein by reference.

Thus the microbial host cells of the invention can be used to produce compounds of interest, in particular VHHs, such as the VHHs disclosed herein, as well as other VHHs, such as those disclosed in WO2014/177595 or WO2014/191146. In some embodiments, the VHHs are fused to a carrier peptide.

The microbial host cells used in the invention are capable of expressing a compound of interest.

With “capable of expressing a compound of interest” it is meant that the microbial host cell is modified in such a way that it contains the genetic information of a compound of interest that is under control of a promoter sequence that drives the expression of said compound either in a continuous manner or during conditions suitable for expression. For example, generally the microbial host cell may comprise a polynucleotide coding for the compound of interest. The polynucleotide may be in the form of a plasmid or a vector. The polynucleotide may be introduced into the microbial host cell according to any suitable method known to the skilled person. For example, the polynucleotide may be introduced into the cell by transformation, for example protoplast-mediated transformation (PMT), Agrobacterium-mediated transformation (AMT), electroporation, biolistic transformation (particle bombardment), or shock-wave-mediated transformation (SWMT). The compound of interest is therefore a recombinant or heterologous compound of interest, since it is not encoded by the wild-type genome of the microbial host cell.

The compound of interest may be under the control of (i.e. may be operably linked to) a promoter sequence. The promoter sequence may promote the expression of the compound of interest in and by the microbial host cell. In some embodiments, the compound of interest may be operably linked to a constitutive promoter, or the compound of interest may be operably linked to an inducible promoter. When linked to a inducible promoter, methods of the invention may comprise a step of inducing expression of the compound of interest by the microbial host cell.

With a “promoter sequence” it is meant a nucleotide sequence that is preferably recognized by a polypeptide, for example a regulator of transcription or at the very least allows the correct formation of a RNA-polymerase complex in such a way that expression of a compound of interest, of which the polynucleotide is located downstream of the promoter sequence as is well known in the art, is established in a continuous manner or during conditions suitable for expression, as to produce the compound of interest or a compound involved in the production of the compound of interest. The promoters are generally promoters that are functional in fungi. These promoters can be but are not limited to alcA Alcohol dehydrogenase I, amyB TAKA-amylase A, bli-3 Blue light-inducible gene, bphA Benzoate p-hydrolase, catR Catalase, cbhI Cellobiohydrolase I, cbh2 cellobiohydrolase 2, cel5a endoglucanase 2, cel12a endogluconase 3, cre1 Glucose repressor, exylA endoxylanase, gas 1,3-beta-glucanosyltransferase, glaA Glucoamylase A, gla1 Glucoamylase, mir1 Siderophore transporter, niiA Nitrite reductase, qa-2 Catabolic 3-dehydroquinase, Smxyl endoxylanase, tcu-1 Copper transporter, thiA thiamine thiazole synthase, vvd Blue light receptor, xyl1 endoxylanase, xylP endoxylanase, xyn1 endoxylanase 1, xyn2 endoxylanase 2, xyn3 endoxylanase 3, zeaR regulator of transcription, cDNA1, eno1 enolase, gpd1 glyceraldehyde-3-phosphate dehydrogenase, pdc1 pyruvate decarboxylase, pki1 pyruvate kinase, tef1 transcription elongation factor 1α, rp2 ribosomal protein, stp1 sugar transporter or tauD3 tauD like dioxygenase.

As used herein, the terms “polypeptide”, “protein”, “peptide”, and “amino acid sequence” are used interchangeably, and refer to a polymeric form of amino acids of any length, which can include coded and non-coded amino acids, chemically or biochemically modified or derivatized amino acids, and polypeptides having modified peptide backbones.

In some embodiments the compound of interest is a polypeptide that is fused to a second polypeptide and where the second polypeptide is a “carrier peptide”. Carrier peptides are peptides that may be produced and secreted by the microbial host cell. Carrier peptides may be abundant or produced in quantities that exceed other peptides not suitable to be used as a carrier peptide. Carrier peptides may be native to the microbial host cell. Thus, carrier peptides may serve to increase the production and/or the secretion of the compound of interest as compared to the production and/or secretion of a compound of interest not fused to a carrier peptide. Carrier peptides may be, but are not limited to, a glucoamylase Gla peptide, a cellobiohydrolase Cbh1 peptide or a cellobiohydrolase Cbh2 peptide. Carrier peptides may consist of a functional fragment of, but not limited to, glucoamylase Gla peptide, a cellobiohydrolase Cbh1 peptide or a cellobiohydrolase Cbh2 peptide. A functional fragment of a carrier peptide may be limited to the N-terminal region of, but not limited to, glucoamylase Gla peptide, a cellobiohydrolase Cbh1 peptide or a cellobiohydrolase Cbh2 peptide. Alternatively the functional fragment of a carrier peptide may be limited to the catalytic domain of the carrier peptide, such as the catalytic domain of the Cbh1 carrier peptide. The N-terminal region may consist of only the signal peptide or signal sequence of, but not limited to glucoamylase Gla peptide, a cellobiohydrolase Cbh1 peptide or a cellobiohydrolase Cbh2 peptide. The signal peptide or signal sequence may allow for the secretion of the compound of interest. In more preferred embodiments the carrier peptide is fused to the N-terminus of the compound of interest. In some embodiments the compound of interest and the carrier peptide may be separated by a proteolytic cleavage site. That is to say, a third peptide containing a proteolytic cleavage site can be present between the compound of interest and the carrier peptide. In a more preferred embodiment the proteolytic cleavage site is fused to the C-terminus of the carrier-peptide and the N-terminus of the compound of interest. Thus, the polypeptide may be a fusion protein comprising, in a 5′ to 3′ order, a carrier peptide, a proteolytic cleavage sate, and the compound of interest. The proteolytic cleavage site may be, but is not limited to, the KexB proteolytic cleavage site. The presence of a proteolytic cleavage site allows for the compound of interest to be separated from the carrier peptide by action of a protease. This protease may be but is not limited to the KexB protease. In some embodiments this separation takes place at the time of secretion or immediately after secretion of the fusion protein. In other embodiments the protease separating the compound of interest can be added to the fermentation medium. In some embodiments the protease separating the compound of interest can be added during or after purification of the fusion protein. In a preferred embodiment the separation of the compound of interest from the carrier peptide can occur by protease activity native to the microbial host cell.

As used herein, the terms “nucleic acid molecule”, “polynucleotide”, “polynucleic acid”, “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural similarity between two macromolecules, particularly between two polypeptides or polynucleotides, from same or different taxons, wherein said similarity is due to shared ancestry. Hence, the term “homologues” denotes so-related macromolecules having said secondary and optionally tertiary structural similarity.

For comparing two or more nucleotide sequences, sequence “identity” may be used, in which the “(percentage of) sequence identity” between a first nucleotide sequence and a second nucleotide sequence may be calculated using methods known by the person skilled in the art.

The terms “sequence identity”, “% identity” are used interchangeable herein. For the purposes of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared.

Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package may be used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/.

For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids over their entire length, they are identical or have 100% identity. Amino acid sequences and nucleic acid sequences are said to be “exactly the same” or “identical” if they have 100% sequence identity over their entire length.

In determining the degree of sequence identity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art.

As used herein, the term “antibody” refers to polyclonal antibodies, monoclonal antibodies, humanized antibodies, chimeric antibodies, minibodies, diabodies, nanobodies, nanoantibodies, affibodies, alphabodies, designed ankyrin-repeat domains, anticalins, knottins, engineered CH2 domains, single-chain antibodies, or fragments thereof such as Fab F(ab)₂, F(ab)₃, scFv, a single domain antibody, a heavy chain variable domain of an antibody, a heavy chain variable domain of a heavy chain antibody (VHH), the variable domain of a camelid heavy chain antibody, a variable domain of the a new antigen receptor (vNAR), a variable domain of a shark new antigen receptor, or other fragments or antibody formats that retain the antigen binding function of a parent antibody. As such, an antibody may refer to an immunoglobulin, or fragment or portion thereof, or to a construct comprising an antigen-binding portion comprised within a modified immunoglobulin-like framework, or to an antigen-binding portion comprised within a construct comprising a nonimmunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab)2, Fv, and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies. As used herein, the term “polyclonal antibody” refers to an antibody composition having a heterogeneous antibody population. Polyclonal antibodies are often derived from the pooled serum from immunized animals or from selected humans.

“Heavy chain variable domain of an antibody or a functional fragment thereof” (also indicated hereafter as VHH), as used herein, means (i) the variable domain of the heavy chain of a heavy chain antibody, which is naturally devoid of light chains, including but not limited to the variable domain of the heavy chain of heavy chain antibodies of camelids or sharks or (ii) the variable domain of the heavy chain of a conventional four-chain antibody (also indicated hereafter as VH), including but not limited to a camelized (as further defined herein) variable domain of the heavy chain of a conventional four-chain antibody (also indicated hereafter as camelized VH).

As used herein, the terms “complementarity determining region” or “CDR” within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains.

As further described hereinbelow, the amino acid sequence and structure of a heavy chain variable domain of an antibody can be considered, without however being limited thereto, to be comprised of four framework regions or “FR's”, which are referred to in the art and hereinbelow as “framework region 1” or “FR1”; as “framework region 2” or “FR2”; as “framework region 3” or “FR3”; and as “framework region 4” or “FR4”, respectively, which framework regions are interrupted by three complementary determining regions or “CDR's”, which are referred to in the art as “complementarity determining region 1” or “CDR1”; as “complementarity determining region 2” or “CDR2”; and as “complementarity determining region 3” or “CDR3”, respectively.

As also further described hereinbelow, the total number of amino acid residues in a heavy chain variable domain of an antibody (including a VHH or a VH) can be in the region of 110-130, is preferably 112-115, and is most preferably 113. It should however be noted that parts, fragments or analogs of a heavy chain variable domain of an antibody are not particularly limited as to their length and/or size, as long as such parts, fragments or analogs retain (at least part of) the functional activity, such as the pesticidal, biocidal, biostatic activity, fungicidal or fungistatic activity (as defined herein) and/or retain (at least part of) the binding specificity of the original a heavy chain variable domain of an antibody from which these parts, fragments or analogs are derived from. Parts, fragments or analogs retaining (at least part of) the functional activity, such as the pesticidal, biocidal, biostatic activity, fungicidal or fungistatic activity (as defined herein) and/or retaining (at least part of) the binding specificity of the original heavy chain variable domain of an antibody from which these parts, fragments or analogs are derived from are also further referred to herein as “functional fragments” of a heavy chain variable domain.

A method for numbering the amino acid residues of heavy chain variable domains is the method described by Chothia et al. (Nature 342, 877-883 (1989)), the so-called “AbM definition” and the so-called “contact definition”. Herein, this is the numbering system adopted.

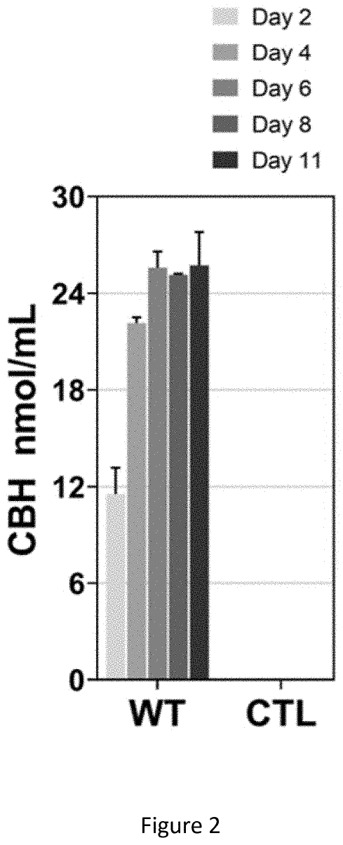

Alternatively, the amino acid residues of a variable domain of a heavy chain variable domain of an antibody (including a VHH or a VH) may be numbered according to the general numbering for heavy chain variable domains given by Kabat et al. (“Sequence of proteins of immunological interest”, US Public Health Services, NIH Bethesda, Md., Publication No. 91), as applied to VHH domains from Camelids in the article of Riechmann and Muyldermans, referred to above (see for example FIG. 2 of said reference).

For a general description of heavy chain antibodies and the variable domains thereof, reference is inter alia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx NV; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by Ablynx; Hamers-Casterman et al., Nature 1993 Jun. 3; 363 (6428): 446-8.

Generally, it should be noted that the term “heavy chain variable domain” as used herein in its broadest sense is not limited to a specific biological source or to a specific method of preparation. For example, as will be discussed in more detail below, the heavy chain variable domains of the invention can be obtained (1) by isolating the VHH domain of a naturally occurring heavy chain antibody; (2) by isolating the VH domain of a naturally occurring four-chain antibody (3) by expression of a nucleotide sequence encoding a naturally occurring VHH domain; (4) by expression of a nucleotide sequence encoding a naturally occurring VH domain (5) by “camelization” (as described below) of a naturally occurring VH domain from any animal species, in particular a species of mammal, such as from a human being, or by expression of a nucleic acid encoding such a camelized VH domain; (6) by “camelisation” of a “domain antibody” or “Dab” as described by Ward et al (supra), or by expression of a nucleic acid encoding such a camelized VH domain (7) using synthetic or semi-synthetic techniques for preparing proteins, polypeptides or other amino acid sequences; (8) by preparing a nucleic acid encoding a VHH or a VH using techniques for nucleic acid synthesis, followed by expression of the nucleic acid thus obtained; and/or (9) by any combination of the foregoing. Suitable methods and techniques for performing the foregoing will be clear to the skilled person based on the disclosure herein and for example include the methods and techniques described in more detail hereinbelow.

However, according to a specific embodiment, the heavy chain variable domains as disclosed herein do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

The term “affinity”, as used herein, refers to the degree to which a polypeptide, in particular an immunoglobulin, such as an antibody, or an immunoglobulin fragment, such as a VHH, binds to an antigen so as to shift the equilibrium of antigen and polypeptide toward the presence of a complex formed by their binding. Thus, for example, where an antigen and antibody (fragment) are combined in relatively equal concentration, an antibody (fragment) of high affinity will bind to the available antigen so as to shift the equilibrium toward high concentration of the resulting complex. The dissociation constant is commonly used to describe the affinity between the protein binding domain and the antigenic target. Typically, the dissociation constant is lower than 10-5 M. Preferably, the dissociation constant is lower than 10-6 M, more preferably, lower than 10-7 M. Most preferably, the dissociation constant is lower than 10-8 M.

The terms “specifically bind” and “specific binding”, as used herein, generally refers to the ability of a polypeptide, in particular an immunoglobulin, such as an antibody, or an immunoglobulin fragment, such as a VHH, to preferentially bind to a particular antigen that is present in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold).

Accordingly, an amino acid sequence as disclosed herein is said to “specifically bind to” a particular target when that amino acid sequence has affinity for, specificity for and/or is specifically directed against that target (or for at least one part or fragment thereof).

The “specificity” of an amino acid sequence as disclosed herein can be determined based on affinity and/or avidity.

An amino acid sequence as disclosed herein is said to be “specific for a first target antigen of interest as opposed to a second target antigen of interest” when it binds to the first target antigen of interest with an affinity that is at least 5 times, such as at least 10 times, such as at least 100 times, and preferably at least 1000 times higher than the affinity with which that amino acid sequence as disclosed herein binds to the second target antigen of interest. Accordingly, in certain embodiments, when an amino acid sequence as disclosed herein is said to be “specific for” a first target antigen of interest as opposed to a second target antigen of interest, it may specifically bind to (as defined herein) the first target antigen of interest, but not to the second target antigen of interest.

“Fungicidal activity”, as used herein, means to interfere with the harmful activity of a fungus, including but not limited to killing the fungus, inhibiting the growth or activity of the fungus, altering the behavior of the fungus, and repelling or attracting the fungus.

“Fungistatic activity”, as used herein, means to interfere with the harmful activity of a fungus, including but not limited to inhibiting the growth or activity of the fungus, altering the behavior of the fungus, and repelling or attracting the fungus.

“Culturing”, “cell culture”, “fermentation”, “fermenting” or “microbial fermentation” as used herein means the use of a microbial cell to produce a compound of interest, such as a polypeptide, at an industrial scale, laboratory scale or during scale-up experiments. It includes suspending the microbial cell in a broth or growth medium, providing sufficient nutrients including but not limited to one or more suitable carbon source (including glucose, sucrose, fructose, lactose, Avicel®, xylose, galactose, ethanol, methanol, or more complex carbon sources such as molasses or wort), nitrogen source (such as yeast extract, peptone or beef extract), trace element (such as iron, copper, magnesium, manganese or calcium), amino acid or salt (such as sodium chloride, magnesium chloride or natrium sulfate) or a suitable buffer (such as phosphate buffer, succinate buffer, HEPES buffer, MOPS buffer or Tris buffer). Optionally it includes one or more inducing agents driving expression of the compound of interest or a compound involved in the production of the compound of interest (such as lactose, IPTG, ethanol, methanol, sophorose or sophorolipids). If can also further involve the agitation of the culture media via for example stirring of purging to allow for adequate mixing and aeration. It can further involve different operational strategies such as batch cultivation, semi-continuous cultivation or continuous cultivation and different starvation or induction regimes according to the requirements of the microbial cell and to allow for an efficient production of the compound of interest or a compound involved in the production of the compound of interest. Alternatively, the microbial cell is grown on a solid substrate in an operational strategy commonly known as solid state fermentation.

“Cultured under conditions conducive to the expression of a compound of interest” means the microbial cell is cultured in such a way that the microbial host cell produces the compound of interest. Production is caused by transcription of the polynucleotide encoding the compound of interest into mRNA, followed by translation of the mRNA into a protein, wherein the protein is the compound of interest. Such conditions may vary according to the microbial cell and/or the polynucleotide being used. For example. as noted elsewhere, the nucleotide encoding the compound of interest may be under the control of an inducible promoter. In such embodiments, “conditions conducive to the expression of a compound of interest” would include induction of the promoter to cause expression of the gene encoding the compound of interest to cause production of said compound. If the nucleotide encoding the compound of interest is be under the control of a constitutive promoter, “conditions conducive to the expression of a compound of interest” may not require anything further than conditions that allow for microbial host cell growth.

Fermentation broth, culture media or cell culture media as used herein can mean the entirety of liquid or solid material of a fermentation or culture at any time during or after that fermentation or culture, including the liquid or solid material that results after optional steps taken to isolate the compound of interest. As such, the fermentation broth or culture media as defined herein includes the surroundings of the compound of interest after isolation of the compound of interest, during storage and/or during use as an agrochemical or pharmaceutical composition. Fermentation broth is also referred to herein as a culture medium or cell culture medium.

“Peptone” as used herein means a “protein hydrolysate”, which is any water-soluble mixture of polypeptides and amino acids formed by the partial hydrolysis of protein. More specifically “peptone” or “protein hydrolysate” are the water-soluble products derived from the partial hydrolysis of protein rich starting material which can be derived from plant, yeast, or animal sources. Typically, “peptone” or “protein hydrolysate” are produced by a protein hydrolysis process accomplished using strong acids, bases, or proteolytic enzymes. In more detail peptone or protein hydrolysates are produced by combining protein and demineralized water to form a thick suspension of protein material in large-capacity digestion vessels, which are stirred continuously throughout the hydrolysis process. For acid or basic hydrolysis, the temperature is adjusted, and the digestion material added to the vessel. For proteolytic digestion, the protein suspension is adjusted to the optimal pH and temperature for the specific enzyme or enzymes chosen for the hydrolysis. The desired degree of hydrolysis depends on the amount of enzyme, time for digestion, and control of pH and temperature. A typical “peptone” or “protein hydrolysate” may comprise about 25% polypeptides, about 30% free amino acids, about 20% carbohydrates, about 15% salts and trace metals and about 10% vitamins, organic acids, and organic nitrogen bases. Depending on the starting material “peptone” or “protein hydrolysate” can be completely free of animal derived products and/or GMO products. For example, “Peptone” or “protein hydrolysate” can be produced using high quality pure protein as a starting material. Alternatively, “peptone” or “protein hydrolysate” can be produced by using soymeal as a starting material. When soymeal is used as a starting material this soymeal can be free of animal sources. This soymeal can furthermore be free of GMO material. This soymeal can be defatted soymeal. Alternatively, “peptone” or “protein hydrolysate” can be produced by using casein as a starting material. Alternatively, “peptone” or “protein hydrolysate” can be produce by using milk as a starting material. Alternatively, “peptone” or “protein hydrolysate” can be produce by using meat paste as a starting material. When meat paste is used as a starting material this meat paste can be for example from bovine or porcine origin. When meat paste is used as a starting material this meat paste can be derived from organs, such as harts or alternatively for example muscle tissue. Alternatively, “peptone” or “protein hydrolysate” can be produced using gelatin as a starting material. Alternatively, “peptone” or “protein hydrolysate” can be produced by using yeast as a starting material.

Accordingly, in some embodiments, the peptone is the product of partial hydrolysis of plant, animal or yeast protein.

In some embodiments, the peptone is produced by acid hydrolysis, by base hydrolysis or by enzymatic digestion.

In some embodiments, the peptone comprises at least about 5% polypeptides (weight/weight %). For example, in some embodiments the peptone comprises from about 5% to about 50% (weight/weight %) polypeptides.

In some embodiments, the peptone comprises at least about 5% (weight/weight %) free amino acids.

For example, in some embodiments the peptone comprises from about 5% to about 50% (weight/weight %) free amino acids.

In some embodiments, the peptone comprises at least about 5% (weight/weight %) salts. For example, in some embodiments the peptone comprises from about 5% to about 20% (weight/weight %) salts.

In some embodiments, the peptone comprises at least about 5% (weight/weight %) carbohydrates.

For example, in some embodiments the peptone comprises from about 5% to about 40% (weight/weight %) carbohydrates.

In some embodiments, the peptone comprises at least about 5% (weight/weight %) carbohydrates about 5% (weight/weight %) vitamins, organic acids, and organic nitrogen bases. For example, in some embodiments the peptone comprises from about 5% to about 20% (weight/weight %) vitamins, organic acids, and organic nitrogen bases.

In some embodiments, the peptone comprises from at least about 5% (weight/weight %) of polypeptides, at least about 5% (weight/weight %) free amino acids, at least about 5% (weight/weight %) salts, at least about 5% (weight/weight %) carbohydrates and at least about 5% (weight/weight %) in total of vitamins, organic acids, and organic nitrogen bases.

In some embodiments, the peptone comprises from about 15% to about 35% (weight/weight %) polypeptides, from about 20% to about 40% (weight/weight %) free amino acids, from about 10% to about 30% (weight/weight %) carbohydrates, from about 5% to about 25% (weight/weight %) salts, and from about 5% to about 15% (weight/weight %) in total of vitamins, organic acids, and organic nitrogen bases. Of course the skilled person will be aware the total amount cannot exceed 100% when all components are added together. The peptone may comprise additional components not specifically listed here.

In some embodiments, the peptone is free of animal derived products.

In some embodiments, the peptone is the product of partial hydrolysis of soymeal, casein, milk, meat, gelatine, or yeast.

Culturing in the presence of peptone means the cell culture medium comprise peptone. The peptone may be present at any suitable concentration. For example, in some embodiments the peptone concentration may be from about 1 g/L to about 100 g/L, for example from about 10 g/L to about 80 g/L, for example about 20 g/L, about 30 g/L, about 40 g/L, about 50 g/L, about 60 g/L, or about 70 g/L.

The cell culture medium used for culture of the microbial host cell may already comprise peptone. Alternatively, the cell culture medium may be modified to include peptone. For example, peptone may be added to the cell culture medium at any suitable time during the culturing of the microbial cell. For example, in embodiments where the compound of interest is encoded by a nucleotide that is operably linked to an inducible promoter, the peptone may be added to the cell culture medium in the fermentation chamber at the same time as or shortly after expression of the compound of interest is induced. Alternatively, the peptone may be added to the cell culture medium in the fermentation chamber before induction of expression of the compound of interest.

In embodiments where the cell culture medium does not already comprise peptone and this must be added to the cell culture medium, this may be added to the cell culture medium before adding the cell culture medium to the fermentation chamber. Alternatively, the peptone may be added to the fermentation chamber separately, preferably after the cell culture medium is added to the fermentation chamber.

“Isolating the compound of interest” is an optional step or series of steps taking the cell culture media or fermentation broth as an input and increasing the amount of the compound of interest relative to the amount of culture media or fermentation broth. Isolating the compound of interest may alternatively or additionally comprises obtaining or removing the compound of interest form the culture media or fermentation broth. Isolating the compound of interest can involve the use of one or multiple combinations of techniques well known in the art, such as precipitation, centrifugation, sedimentation, filtration, diafiltration, affinity purification, size exclusion chromatography and/or ion exchange chromatography. In some embodiments, isolating the compound of interest may comprise a step of lysing the microbial cells to release the compound of interest, for example if the compound of interest is not secreted by the microbial cells, or at least is not secreted by the microbial cells to a significant enough degree. Isolating the compound of interest may be followed by formulation of the compound of interest into an agrochemical or pharmaceutical composition.

The term “yield” as used herein refers to the amount of a compound of interest produced. When using the term “improved” or “increased” or a similar term when referring to “yield”, it is meant that the compound of interest produced by the microbial host cell of the invention capable of producing a compound of interest is increased in quantity, quality, stability and/or concentration either in the fermentation broth or cell culture media, as a purified or partially purified compound, during storage and/or during use as an agrochemical or pharmaceutical composition. The increase in yield is compared to the yield of compound of interest produced by a microbial host cell that has been cultured in the absence of peptone.

In some embodiments, the yield is increased by at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% or at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290% or at least about 300%, at least about 500%, at least about 1000% or at least about 1500% when a microbial host cell is cultured in the presence of peptone, compared to when the microbial host cell is cultured in the absence of peptone. In some embodiments, the yield of the compound of interest may be at least about 5 g/L. For example, the yield of the compound of interest may be from about 5 g/L to about 50 g/L or more. In some embodiments, the yield of the compound of interest may be from about 10 g/L to about 50 g/L, from about 15 g/L to about 50 g/L or from about 20 g/L to about 50 g/L. Yield of the compound of interest may also be referred to as the titer of the compound of interest. The yield or titer may be determined according to the amount of compound of interest in the supernatant (or cell culture medium), the amount of compound of interest comprised with cellular material, or a combination of both (the amount of compound of interest in the supernatant (or cell culture medium) and the amount of compound of interest comprised with cellular material).

“Agrochemical”, “agrochemically” or “agrochemically suitable” as used herein, means suitable for use in the agrochemical industry (including agriculture, horticulture, floriculture and home and garden uses), but also products intended for non-crop related uses such as public health/pest control operator uses to control undesirable insects and rodents, household uses, such as household fungicides and insecticides and agents, for protecting plants or parts of plants, crops, bulbs, tubers, fruits (e.g. from harmful organisms, diseases or pests); for controlling, preferably promoting or increasing, the growth of plants; and/or for promoting the yield of plants, crops or the parts of plants that are harvested (e.g. its fruits, flowers, seeds etc.). Examples of such substances will be clear to the skilled person and may for example include compounds that are active as insecticides (e.g. contact insecticides or systemic insecticides, including insecticides for household use), herbicides (e.g. contact herbicides or systemic herbicides, including herbicides for household use), fungicides (e.g. contact fungicides or systemic fungicides, including fungicides for household use), nematicides (e.g. contact nematicides or systemic nematicides, including nematicides for household use) and other pesticides or biocides (for example agents for killing insects or snails); as well as fertilizers; growth regulators such as plant hormones; micro-nutrients, safeners, pheromones; repellants; insect baits; and/or active principles that are used to modulate (i.e. increase, decrease, inhibit, enhance and/or trigger) gene expression (and/or other biological or biochemical processes) in or by the targeted plant (e.g. the plant to be protected or the plant to be controlled), such as nucleic acids (e.g., single stranded or double stranded RNA, as for example used in the context of RNAi technology) and other factors, proteins, chemicals, etc. known per se for this purpose, etc. Examples of such agrochemicals will be clear to the skilled person; and for example include, without limitation: glyphosate, paraquat, metolachlor, acetochlor, mesotrione, 2,4-D, atrazine, glufosinate, sulfosate, fenoxaprop, pendimethalin, picloram, trifluralin, bromoxynil, clodinafop, fluroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof.

An “agrochemical composition”, as used herein means a composition for agrochemical use, as further defined, comprising at least one active substance, optionally with one or more additives (for example one or more additives favoring optimal dispersion, atomization, deposition, leaf wetting, distribution, retention and/or uptake of agrochemicals). It will become clear from the further description herein that an agrochemical composition as used herein includes biological control agents or biological pesticides (including but not limited to biological biocidal, biostatic, fungistatic and fungicidal agents) and these terms will be interchangeably used in the present application. Accordingly, an agrochemical composition as used herein includes compositions comprising at least one biological molecule as an active ingredient, substance or principle for controlling pests in plants or in other agro-related settings (such for example in soil). Non-limiting examples of biological molecules being used as active principles in the agrochemical compositions disclosed herein are proteins (including antibodies and fragments thereof, such as but not limited to heavy chain variable domain fragments of antibodies, including VHH's), nucleic acid sequences, (poly-) saccharides, lipids, vitamins, hormones glycolipids, sterols, and glycerolipids. As a non-limiting example, the additives in the agrochemical compositions disclosed herein may include but are not limited to excipients, diluents, solvents, adjuvants, surfactants, wetting agents, spreading agents, oils, stickers, thickeners, penetrants, buffering agents, acidifiers, anti-settling agents, anti-freeze agents, photo-protectors, defoaming agents, biocides and/or drift control agents. The compound of interest may be formulated with one or more such components when preparing an agrochemical composition. For example, the compound of interest may be formulated with one or more additives, for example one or more agrochemically acceptable excipients.

A “pharmaceutical composition”, “pharmaceutically” or “pharmaceutically suitable” as used herein means a composition for medical use. For example, the composition may be suitable for injection or infusion which can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin. The compound of interest may be formulated with one or more such components when preparing a pharmaceutical composition. For example, the compound of interest may be formulated with one or more additives, for example one or more pharmaceutically acceptable excipients.

The methods of the invention may comprise a step of producing the microbial host cell. The methods may comprise inserting a polynucleotide coding for a compound of interest into the microbial host cell. Therefore, the methods may begin from a parental microbial cell, and include a step of inserting a polynucleotide coding for a compound of interest into the parental microbial cell to provide the microbial host cell.

### Method of Making Compounds of Interest

The invention provides methods for the production of a compound of interest. The compound of interest may be a compound as described herein, for example an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor, a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH. The methods comprise providing a microbial host cell of the invention, which is capable of expressing the compound of interest. The method further comprises culturing said microbial host cell under conditions conducive to the expression of the compound of interest, wherein the host cell is cultured in the present of peptone. The method may further optionally comprise a step of isolating the compound of interest from the culture medium or fermentation broth.

The microbial host cell that is provided may already be capable of expressing the compound of interest. For example, the microbial host cell may be provided already comprising a polynucleotide coding for the compound of interest, and the sequence encoding the compound of interest may be operable linked to a promoter (for example a constitutive promoter or an inducible promoter). Alternatively, the method may comprise a step of transforming a parental microbial cell with the polynucleotide to insert the polynucleotide into the microbial cell.

In some embodiments, the methods may comprise a step of inducing expression of the compound of interest by the microbial host cell. For example, if the compound of interest is encoded by a nucleotide sequence that is operably linked to an inducible promoter, the method may comprise a step of inducing the expression of the compound of interest. A common inducible promoter that may be used is the inducible cbh1 or cbh2 promoter, in which administration of lactose will initiate expression. Other inducible promoters could of course be used. If the sequence encoding the compound of interest is under the control of a constitutive promoter, no specific step of induction of expression may be required.

Fermentation or culture of the microbial host cells may occur in a solid fermentation or culture setting or a liquid fermentation or culture setting. Solid-state fermentation or culture may comprise seeding the microbial host cell on a solid culture substrate, and methods of solid-state fermentation or culture are known the skilled person. Liquid fermentation or culture may comprise culturing the microbial host cell in a liquid cell culture medium.

The method may also comprise a step of isolating the compound of interest produced by the microbial host cell, for example isolating the compound of interest from the fermentation broth or cell culture medium.

The method may further comprise a step of formulating the compound of interest into a agrochemical or pharmaceutical composition. The step of formulating the compound of interest into an agrochemical composition may comprise formulating the compound of interest with one or more agrochemically acceptable excipients. The step of formulating the compound of interest into a pharmaceutical composition may comprise formulating the compound of interest with one or more pharmaceutically acceptable excipients.

The present invention therefore provides compounds of interest obtained by a method of the present invention. The present invention also therefore provides an agrochemical or pharmaceutical composition obtained by a method of the present invention.

The present invention also provides the use of peptone as a yield increasing agent in a method of production of a compound of interest, wherein the method comprises:
providing a microbial host cell capable of expressing the compound of interest;
culturing said microbial host cell under conditions conducive to the expression of a compound of interest, wherein the microbial host is cultured in the presence of peptone.

Use as a yield increasing agent means the peptone is used to increase the yield of a compound of interest produced by a microbial cell comprising a polynucleotide encoding the compound of interest. The increase is the increase in yield compared to when the microbial cell comprising a polynucleotide encoding the compound of interest is cultured under conditions conducive to the expression of a compound of interest and in the presence of peptone, compared to the yield when the microbial cell comprising a polynucleotide encoding the compound of interest is cultured under conditions conducive to the expression of a compound of interest and in the absence of peptone.

The present invention also provides the use of a microbial host cell for the production of a compound of interest, wherein the microbial host cell comprises at least one polynucleotide coding for the compound of interest, and wherein the method comprises culturing the microbial host cell under conditions conducive to the expression of the compound of interest, wherein the microbial host is cultured in the presence of peptone.

Any methods comprising or requiring the culturing or fermentation of the microbial host cell comprise the culture or fermentation of the host cell is a suitable medium. Generally, the medium will comprise any and all nutrients required for the microbial host cell to grow. The skilled person will be aware of the required components of the cell culture media or fermentation broth, which may differ depending on the species of microbial host cell being cultured. The cell culture media or fermentation broth comprises a nitrogen source, specifically peptone.

In some embodiments, the cell culture medium may comprise ammonium. In some preferred embodiments, the cell culture does not comprise ammonium.

In some embodiments, the sole source of nitrogen in the cell culture medium is peptone.

In some embodiments, the cell culture contains both ammonium and peptone as a source of nitrogen.

The present invention also provides methods for improving the yield of a compound of interest, comprising providing a microbial cell culture comprising a microbial host cell capable of expressing the compound of interest and a cell culture medium, and adding peptone to the cell culture medium. Alternatively, the method may comprise adding peptone to the cell culture medium, and subsequently using the cell culture medium for culture of the microbial host cells. Such methods may be further defined according to the other aspects of the invention discussed herein.

### Kit of Parts

The present invention also provides a kit of parts. The kit comprises peptone, and a microbial host cell.

The host cell may be capable of expressing a compound of interest. For example, the microbial host cell may comprise a polynucleotide coding for the compound of interest. Alternatively or additionally, the kit may further comprise a vector, wherein the vector comprises a polynucleotide sequence coding for the compound of interest. The vector may comprise a promoter that is operable linked to the polynucleotide sequence coding for the compound of interest.

Alternatively, the kit may comprise peptone and a vector, wherein the vector comprises a polynucleotide sequence coding for the compound of interest. The vector may comprise a promoter that is operable linked to the polynucleotide sequence coding for the compound of interest.

Each vector may have one or more antibiotic resistance genes. For example, the vectors may have an antibiotic resistance gene to enable the selection of microbial host cells which have been transformed with the vectors. When the microbial host cell is to be transformed using 2 vectors, the 2 vectors may comprise different antibiotic resistance genes, to enable the selection of microbial host cells that have been transformed with both vectors.

In one embodiment, the kit comprises:
a) peptone
b) a parental microbial cell; and
c) a vector comprising a nucleotide sequence coding for a compound of interest, wherein the nucleotide sequence is operably linked to a promoter.

In one embodiment, the kit comprises:
a) peptone
b) a microbial host cell comprising a polynucleotide coding for a compound of interest, wherein the nucleotide sequence is operably linked to a promoter.

In one embodiment, the kit comprises:
a) peptone
b) a vector comprising a nucleotide sequence coding for a compound of interest, wherein the nucleotide sequence is operably linked to a promoter.

The different components of the kit may each be disposed separately in separate containers.

In some embodiments, the kit may further comprise instructions for use. The instructions may, for example, provide instructions for culturing the microbial host cell to produce a compound of interest using the microbial host cell. The instructions may also alternatively or additionally provide instructions for carrying out any of the methods of the invention disclosed herein.

The vector comprising a nucleotide sequence coding for a compound of interest, if present, is a vector comprising a nucleotide sequence coding for a compound of interest as described elsewhere herein with respect to the modified microbial host cells of the invention.

The Figures and the Experimental Part/Examples are only given to further illustrate the invention and should not be interpreted or construed as limiting the scope of the invention and/or of the appended claims in any way, unless explicitly indicated otherwise herein.

The above disclosure will now be further described by means of the following non-limiting examples.

### EXAMPLES

The following non-limiting Examples describe methods and means according to the invention. Unless stated otherwise in the Examples, all techniques are carried out according to protocols standard in the art. The following examples are included to illustrate embodiments of the invention. Those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Effect of Peptone Addition to Supernatants Containing VHH

To test the effect of adding peptone to the fermentation broth on the degradation of VHH-1 in the extracellular medium, filamentous fungi are grown according to common fermentation protocols, and induction of expression cassettes are done by addition of a suitable inducer (for example lactose). Cells are hereafter removed by centrifugation, filtration, precipitation, or any other suitable method removing cell material. To the resulting supernatant a certain percentage of peptone is added. Different percentages of peptone are used. Furthermore, different kinds of peptone are used to test differences between different protein sources. Examples include peptone from bovine sources or soymeal-based peptone. Together with peptone, a known concentration of pure VHH-1 protein is added to the broth. VHH-1 integrity is monitored over a time-period of up to 14 days. Suitable techniques for assessing VHH-1 concentration and/or integrity are commonly known SDS-PAGE or mass spectrometry techniques such as LC-MS/MS.

### Example 2: Effect of Peptone on VHH Produced from Filamentous Fungi

Filamentous fungi strain containing one or more VHH expression cassettes is grown in a fermentation broth at suitable fermentation conditions and VHH productions and secretion is induced using a suitable inducer. Different percentages of peptone are added to the fermentation broth. Furthermore, different kinds of peptone are used to test differences between different protein sources. Examples include peptone from bovine sources or soymeal-based peptone. Suitable techniques for assessing VHH concentration and/or integrity during and/or at the end of the fermentation are commonly known SDS-PAGE or mass spectrometry techniques such as LC-MS/MS.

### Example 3: Mass Spectrometry Analysis of Fermentation Broth

To gain a better understanding of the effects of different media has on the overall proteome of the fermentation broth non-quantitative LC-MS/MS analyses are performed. Filamentous fungi are grown in standard fermentation conditions and induced. VHH can be produced from an expression cassette from which expression is activated by the inducer, resulting in the secretion of VHH in the extracellular broth. Alternatively, VHH is spiked at the time of induction to stimulate high presence of VHH in extracellular broth. Comparing the proteome of different growth conditions gives an indication of the relative changes in VHH concentration and integrity between the different samples. It also provides information on the relative changes of, for example, certain proteases.

### Example 4: Fermentation Conditions

Example of a general procedure for performing a filamentous fungi fermentation. The fermenter is filled with the appropriate broth. Calibration of the Dissolved oxygen (DO) levels is performed at 37° C., 1200 rpm and 1 lpm of aeration. The pH of the medium in the fermenter is adjusted to around 5 before inoculation of the fermenter. The 5 L fermenter is inoculated on with 1.00% inoculum density in 2000 mL of appropriate broth. Incubation at 28° C.; 1200 rpm and 1 lpm aeration. DO lower limit at 50%. DO cascade output set as 0-50% 1200-1400 rpm of stirrer, 50-100%, 1-10 lpm of aeration. Antifoam Struktol J673-A (Schill und Seilacher) dissolved as 10× in water. Ammonium hydroxide 12.5% as base. Induction is performed with for example with 20% lactose and is initiated after an pO2 spike. The feed rates of the 20% lactose feed is set at 9 mL/h (4.5 mL/l·h).

### Example 5: General Culturing and Fermentation Broth Compositions

In some experiments the culturing or fermentation broth is composed of essentially the following ingredients:

TABLE 1
50x VOGEL'S stock solution

| Vogel's 50x stock solution | Concentration (1 L) | |
|---|---|---|
| Na₃Citrate•2H₂O | 125 | g |
| KH₂PO₄ (anhydrous) | 250 | g |
| (NH₄)₂SO₄ | 100 | g |
| MgSO₄•7H₂O | 10 | g |
| CaCl₂•2H₂O | 5 | g |
| Vogel's Trace Element Solution | 5 | mL |

| | |
|---|---|
| Biotin solution 0.1 mg or 20 mg/mL | 2.5 mL/12.5 μL |

Or alternatively:

| | | |
|---|---|---|
| Na₃Citrate•2H₂O | 125 | g |
| KH₂PO₄ (anhydrous) | 250 | g |
| Peptone | 50 | g |
| MgSO₄•7H₂O | 10 | g |
| CaCl₂•2H₂O | 5 | g |
| Vogel's Trace Element Solution | 5 | mL |

| | |
|---|---|
| Biotin solution 0.1 mg or 20 mg/mL | 2.5 mL/12.5 μL |

TABLE 2
Vogel's trace element solution:

| Vogel's trace element solution | Concentration (1 L) | |
|---|---|---|
| Citric acid | 50 | g |
| ZnSO₄•7H₂O | 50 | g |
| Fe(NH₄)₂SO₄•6H₂O | 10 | g |
| CuSO₄•5H₂O | 2.5 | g |
| MnSO₄•4H₂O | 0.5 | g |
| H₃BO₃ | 0.5 | g |
| Na₂MoO₄•2H₂O | 0.5 | g |

TABLE 3
glucose concentration

| Glucose (50%) | Concentration |
|---|---|
| Glucose | 50% w/v |

TABLE 4
Final medium composition

| Medium composition | 500 mL |
|---|---|
| 50x Vogel's stock solution | 10 mL |
| Glucose (50% stock solution) | 10 mL |
| Yeast extract (80 g/L autoclaved stock) | 47 mL (0.75%) |

### Example 6: Assessing Recombinant Protein Stability

To test the effect of the presence of peptone in the culture broth for the filamentous fungus T. reesei on the stability of VHH-1 (which is a heavy chain variable domain of a heavy chain antibody or a functional fragment thereof) was evaluated in shake flasks with Vogel's medium with peptone or ammonium as nitrogen source prepared as defined in Example 5.

Approximately 5×10⁶ spores/mL of fresh conidia of T. reesei host cell were inoculated into 50 mL of Vogel's liquid medium (with peptone or ammonium as nitrogen source) in 250 mL shake flask in duplicate and incubated at 30° C. An uninoculated control was included in all experiments. After 48 h of growth, 500 μL of a purified VHH-1 (28.61 mg/mL) was spiked into fermentation media and the addition of 1000 μL of 20% lactose inducer was started once a day.

The fermentation broth on day 6 after cellulose induction was sampled and separated by SDS-PAGE electrophoresis to visualize the degradation of VHH-1, taking 30 μL of fermentation broth, 7.5 μL sample buffer and 3.5 μL DTT, and denaturing the samples at 85° C. for 5 min. Note that the samples were immediately transferred to ice before being loaded on SDS-PAGE gels (precast NuPAGE™ 4 to 12%, Bis-Tris, 1.0 mm, Mini Protein Gel, 12-well, Invitrogen).

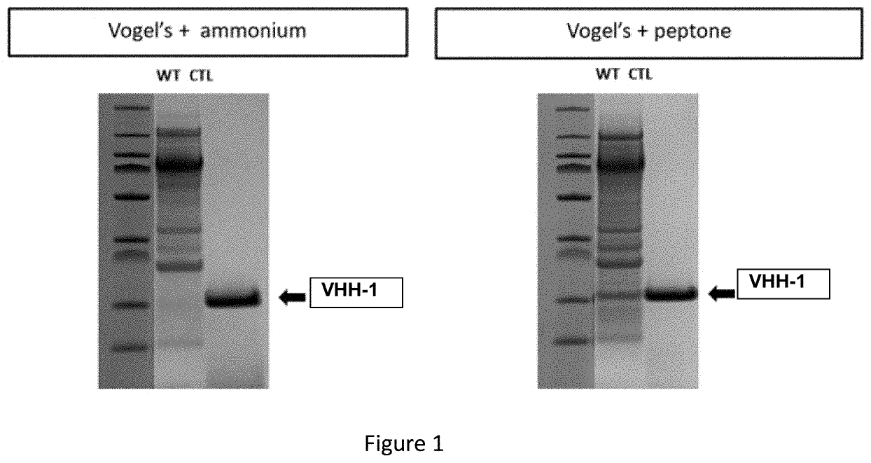

Surprisingly, using Vogel's media with ammonium as a nitrogen source led to the rapid degradation of VHH-1 in all samples as opposed to Vogel's media with peptone as a nitrogen source as shown in FIG. 1 where VHH was still present after 6 days.

### Example 7: Assessing Cellulase Production

Since protein expression can be driven from the cellulase cbhI or cbhII promoters in some cases it could be important that production from these promoters is not impaired in the culture broth. This was evaluated by monitoring changes in cellulase production from shake flasks. Therefore, samples of fermentation broth were analysed by pNP-cellobiohydrolase assay to determine the modified microbial host cell's cellulolytic ability (Coconi Linares et al., 2019).

In Vogel's medium with peptone as the nitrogen source the amount of cellulolytic ability was maintained in the expected range of 24 nmol/ml as shown in FIG. 2.

### Example 8: Effect of Peptone Addition to Fermentation Medium on the Production of Proteases and Cellulases

To investigate the effect of peptone on the production of extracellular proteases and cellulases on a fermenter scale, T. reesei RL-P37 was grown in two different nitrogen sources: peptone, and ammonium. Fed-batch cultivation in 3 L fermenters was evaluated using a defined minimal medium (Trire) with or without 20 g/L peptone, and spiked with 1 mg/L of pure VHH-1. The cultures were induced with 20 g/L lactose at pH 4.8 and incubated at 28° C. for 6 days.

The supernatant was separated after centrifugation and stored at −20° C. The protease quantification assay was performed using the Pierce™ protease colorimetric assay kit (Thermo Scientific). Briefly, 10 μl culture supernatant was diluted with assay buffer and duplicated in two different sets of wells to serve as blanks. Then, 100 μl succinylated casein solution was added to one set of microplate wells and 100 μl assay buffer to the other set. The samples were mixed and incubated for 20 min at 37° C. To analyse casein cleavage, 50 μl of TNBSA reagent was added to every well and the plate was incubated for 20 min at RT. The absorbance at 450 nm was measured for the whole plate. Control wells with supernatant without substrate were used as background controls. The nonspecific background signal was subtracted from specific protease quantification measurements.

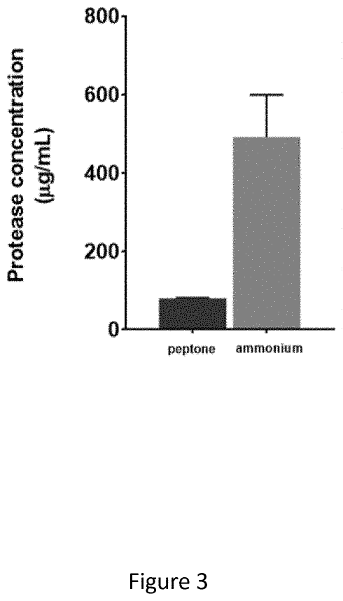

A more than 5-fold decrease in protease concentration was observed in the cultivation with peptone addition as compared to cultivations with ammonia as the sole nitrogen source (FIG. 3). Apparently, the presence of peptone in the culture medium reduces protease activity in the culture medium. These findings showed the importance of the addition of peptone to potentially reduce the degradation of recombinant proteins in fungal hosts.

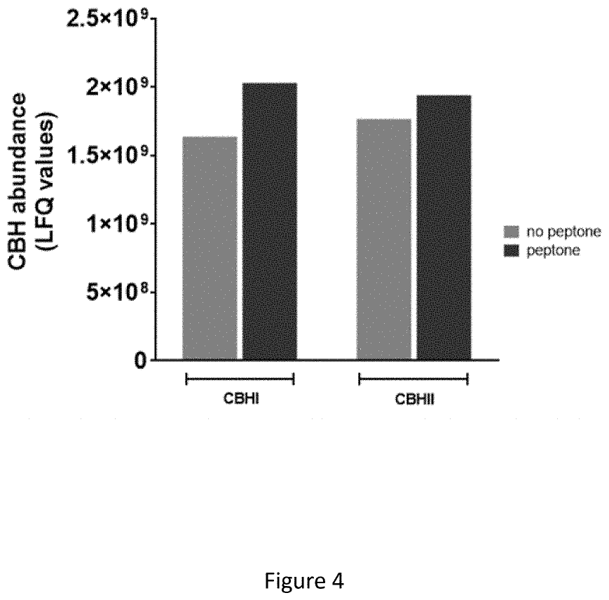

Samples obtained from these fermentation cultures were also TCA-precipitated, digested with trypsin, labelled, and analysed by liquid chromatography-tandem mass spectrometry (LC-MS/MS). MS raw files were imported into MaxQuant and proteins were identified and quantified using the MaxLFQ algorithm to compare the CBHI and CBHII protein abundances between the different samples. The LFQ (label-free quantitation) protein values were normalized to exclude some outliers to best represent the ratio changes of different samples. As shown in FIG. 4 the production of cellulases was increased lightly in the samples where peptone was added to the medium, which indicates that the presence of peptone has a positive effect on cellulases production.

### Example 9: Genomic Integration of a Recombinant Protein Expression Cassette

To express VHH genes in T. reesei, synthetic genes were synthesized comprising a codon-optimized version encoding VHH-1 fused to the cellobiohydrolase I (CBHI) signal peptide and catalytic domain (carrier), with a KexB protease cleavage site in between to release the recombinant protein and carrier protein separately during protein secretion. The CbhI-VHH-1 expression cassette was flanked with 5′ and 3 DNA flanking regions (˜1000 bp each) of the cbhI locus to replace the endogenous cbhI coding region by the VHH expression cassette.

In addition, a selection marker construct was synthesised comprising the hphgene encoding hygromycin phosphotransferase, oliC promoter and the trpC terminator of Aspergillus nidulans (PoliC-hph-TtrpC).

T. reesei was co-transformated with the hph selection marker and the VHH-1 expression cassette targeted to the CbhI locus using a standard poly-ethylene glycol (PEG) mediated transformation method as described previously (Penttila M., Nevalainen, H., Ratto, M., Salminen, E., Knowles, J., 1987. Gene 61, 155-64). Transformation mixtures were plated on PDA plates supplemented with 1.2 M sorbitol and 100 μg/mL of hygromycin and incubated at 28° C. for 4-6 days. Correct integration of the VHH-1 expression cassette in the CbhI locus was confirmed by colony PCR.

### Example 10: Recombinant Protein Expression

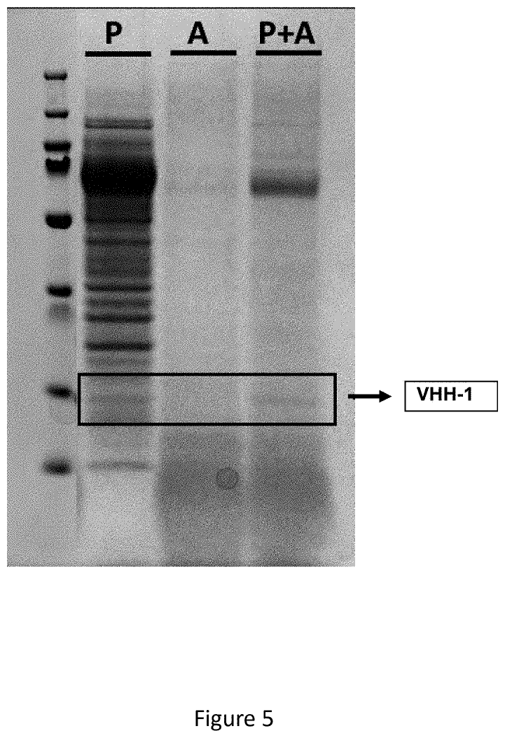

To compare recombinant protein production in medium with and without peptone, the stable VHH-1 transformants were inoculated in minimal medium with either 2% peptone, 2% (NH₄)₂SO₄, or a mixture of 1% peptone and 1% (NH₄)₂SO₄ in shake flasks and incubated for 4 days. The supernatants were collected to quantify the production of recombinant proteins by Bradford. In addition, supernatants were analysed on SDS-PAGE electrophoresis to visualize the production of VHH-1. As shown in FIG. 5 an increase in VHH titer was observed when peptone is included in the medium as compared to cultures without peptone. The increase in VHH-1 titer was observed both in medium with peptone as sole nitrogen source and in medium comprising peptone and ammonium as nitrogen source. These results indicate that addition of peptone improves expression of recombinant protein yields, most likely due to reduced degradation by proteases since protease activity is strongly reduced when using peptone (FIG. 3).

### Embodiments

1. A method for the production of a compound of interest comprising:
   a. providing a microbial host cell comprising at least one polynucleotide coding for a compound of interest;
   b. culturing said microbial host cell under conditions conducive to the expression of the compound of interest, wherein the microbial host is cultured in the presence of peptone.
2. Use of peptone as a yield increasing agent in a method of production of a compound of interest, wherein the method comprises:
   a. providing a microbial host cell capable of expressing the compound of interest;
   b. culturing said microbial host cell under conditions conducive to the expression of a compound of interest, wherein the microbial host is cultured in the presence of peptone.
3. The method or use of embodiment 1 or embodiment 2, wherein the peptone is the product of partial hydrolysis of plant, animal or yeast protein.
4. The method or use of any one of embodiments 1 to 3, wherein the peptone is produced by acid hydrolysis.
5. The method or use of any preceding embodiment, wherein the peptone is produced by base hydrolysis.
6. The method or use of any preceding embodiment, wherein the peptone is produced by enzymatic digestion.
7. The method or use of any preceding embodiment, wherein the peptone comprises from about 5% to about 50% polypeptides.
8. The method or use of any preceding embodiment, wherein the peptone comprises from about 5% to about 50% free amino acids.
9. The method or use of any preceding embodiment, wherein the peptone comprises from about 5% to about 20% salts.
10. The method or use of any preceding embodiment, wherein the peptone comprises from about 5% to about 40% carbohydrates.
11. The method or use of any preceding embodiment, wherein the peptone comprises from about 5% to about 20% vitamins, organic acids, and organic nitrogen bases.
12. The method or use of any preceding embodiment, wherein the peptone comprises from at least about 5% of polypeptides, at least about 5% free amino acids, at least about 5% salts, at least about 5% carbohydrates and at least about 5% in total of vitamins, organic acids, and organic nitrogen bases.
13. The method or use of any preceding embodiment, wherein the peptone comprises from about 15% to about 35% polypeptides, from about 20% to about 40% free amino acids, from about 10% to about 30% carbohydrates, from about 5% to about 25% salts, and from about 5% to about 15% in total of vitamins, organic acids, and organic nitrogen bases.
14. The method of any preceding embodiment, wherein the peptone is free of animal derived products.
15. The method or use of any preceding embodiment, wherein the peptone is the product of partial hydrolysis of soymeal, casein, milk, meat, gelatin, or yeast.
16. The method or use of any preceding embodiment, further comprising isolating a compound of interest from the culture medium.
17. The method or use of any preceding embodiment, wherein the compound of interest is a pharmacologically or agrochemically active polypeptide.
18. The method or use of any preceding embodiment, wherein the yield of the compound of interest is increased by at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290% or at least about 300%, at least about 500%, at least about 1000% or at least about 1500% when compared to when the host cell is cultured in the absence of peptone, optionally wherein the peptone is replaced with an alternative nitrogen source.
19. The method or use to any preceding embodiment, wherein the field of the compound of interest is increased by at least about 100% when compared to when the host cell is cultured in the absence of peptone.
20. The method or use of any preceding embodiment, wherein the method further comprises a step of formulating the compound of interest into a pharmaceutical composition or an agrochemical composition.
21. The method or use of any preceding embodiment, wherein the step of formulation the compound of interest comprises formulation the compound of interest with one or more pharmaceutically acceptable excipients, or one or more agrochemically acceptable excipients.
22. The method or use of any preceding embodiment, wherein the microbial host cell is a eukaryotic host cell.
23. The method or use of embodiment 22, wherein the eukaryotic host cell is a fungal host cell.
24. The method or use of embodiment 22, wherein the eukaryotic host cell is a filamentous fungal host cell.
25. The method or use of any preceding embodiment, wherein the microbial host cell is a cell of a filamentous fungus selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonii, Trichoderma reesei, Penicillium chrysogenum, Penicillium oxalicum and Neurospora crassa.
26. The method or use of embodiment 25, wherein the microbial host cell is a cell of Trichoderma reesei.
27. The method or use of embodiment 25, wherein the microbial host cell is a filamentous fungus selected from the group consisting of Trichoderma reesei QM6a, Trichoderma reesei Rut-C30, Trichoderma reesei RL-P37 and Trichoderma reesei MCG-80.
28. The method or use of embodiment 25, wherein the microbial host cell is a filamentous fungus selected from the group consisting of Myceliophthora heterothallica CBS 131.65, Myceliophthora heterothallica CBS 203.75, Myceliophthora heterothallica CBS 202.75, Myceliophthora heterothallica CBS 375.69 and Myceliophthora heterothallica CBS 663.74.
29. The method or use of any preceding embodiment, wherein the at least one polynucleotide coding for the compound of interest is operably linked to a promoter, optionally to an inducible promoter.
30. The method or use of any preceding embodiment, wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain
31. The method or use of embodiment 30, wherein the compound of interest is an antibody or a functional fragment thereof.
32. The method or use of embodiment 31, wherein the antibody or functional fragment thereof of is a heavy chain variable domain of a heavy chain antibody or a functional fragment thereof (VHH).
33. The method or use of embodiment 32, wherein the VHH is a VHH comprising:
   a. a CDR1 comprising or consisting of a sequence selected from the group consisting of SEQ ID NOs 3, 8 and 13;
   b. a CDR2 comprising or consisting of a sequence selected from the group consisting of SEQ ID NOs: 4, 9 and 14; and
   c. a CDR3 comprising or consisting of a sequence selected from the group consisting of SEQ ID NOs: 5, 10 and 15.
34. The method or use of embodiment 32, wherein the VHH is a VHH comprising:
   a. a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5;
   b. a CDR1 comprising or consisting of the sequence of SEQ ID NO: 8, a CDR2 comprising or consisting of the sequence of SEQ ID NO: 9 and a CDR3 comprising or consisting of the sequence of SEQ ID NO: 10 or
   c. a CDR1 comprising or consisting of the sequence of SEQ ID NO: 13, a CDR2 comprising or consisting of the sequence of SEQ ID NO: 14 and a CDR3 comprising or consisting of the sequence of SEQ ID NO: 15.
35. The method or use of embodiment 32, wherein the VHH is a VHH comprising or consisting of a sequence selected from the group consisting of SEQ ID NOs: 1, 2, 6, 7, 11 and 12.
36. The method or use of embodiment 32, wherein the VHH is a VHH comprising or consisting of SEQ ID NO: 1.
37. The method or use of embodiment 32, wherein the VHH is a VHH comprising or consisting of SEQ ID NO: 2.
38. The method or use of any preceding embodiment, wherein the compound of interest is fused to a carrier peptide.
39. The method or use of any preceding embodiment, wherein the polynucleotide coding for a compound of interest encodes, in a 5′ to 3′ order, a carrier peptide, a proteolytic cleavage side, and the compound of interest.
40. The method or use of any preceding embodiment, wherein the method comprises inserting the polynucleotide coding for a compound of interest into the microbial host cell prior to culturing the host cell.
41. Use of a microbial host cell for the production of a compound of interest, wherein the microbial host comprises at least one polynucleotide coding for the compound of interest, and wherein the method comprises culturing the microbial host cell under conditions conducive to the expression of the compound of interest, wherein the microbial host is cultured in the presence of peptone.
42. The use of a microbial host cell of embodiment 41, wherein the method is the method of any one of embodiments 1 to 40.
43. A kit comprising:
   a. peptone;
   b. a microbial cell; and
   c. a vector comprising a nucleotide sequence coding for a compound of interest, wherein the nucleotide sequence is operably linked to a promoter.
44. A kit comprising:
   a. peptone; and
   b. a microbial cell comprising a nucleotide sequence coding for a compound of interest.
45. The kit of embodiment 43 or embodiment 44, wherein the microbial host cell is a microbial host cell as defined in any one of embodiments 1 to 40.
46. A kit comprising:
   a. peptone
   b. a vector comprising a nucleotide sequence coding for a compound of interest, wherein the nucleotide sequence is operably linked to a promoter.
47. The kit of any one of embodiments 43 to 46, wherein the kit further comprises instructions for use.
48. The kit of any one of embodiments 43 to 47, wherein the components of the kit are disposed separately in different containers.

1. A method for the production of a compound of interest comprising:
a. providing a microbial host cell comprising at least one polynucleotide coding for a compound of interest, wherein the microbial host cell is a filamentous fungal host cell;
b. culturing said microbial host cell under conditions conducive to the expression of the compound of interest, wherein the microbial host is cultured in the presence of peptone
wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain, and
wherein the yield of the compound of interest is increased compared to when the microbial host cell is cultured in the absence of peptone.

2. Use of peptone as a yield increasing agent in a method of production of a compound of interest, wherein the method comprises:
a. providing a microbial host cell capable of expressing the compound of interest, wherein the microbial host cell is a filamentous fungal host cell;
b. culturing said microbial host cell under conditions conducive to the expression of a compound of interest, wherein the microbial host is cultured in the presence of peptone
wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain, and
wherein the yield of the compound of interest is increased compared to when the microbial host cell is cultured in the absence of peptone.

3. The method of claim 1 or the use of claim 2, wherein the peptone is the product of partial hydrolysis of plant, animal or yeast protein.

4. The method or use of any preceding claim, wherein the peptone comprises from about 15% to about 35% polypeptides, from about 20% to about 40% free amino acids, from about 10% to about 30% carbohydrates, from about 5% to about 25% salts, and from about 5% to about 15% in total of vitamins, organic acids, and organic nitrogen bases, optionally wherein the peptone is free of animal derived products.

5. The method or use of any of claims 1 to 4, wherein the peptone is the product of partial hydrolysis of soymeal, casein, milk, meat, gelatin, or yeast.

6. The method or use of any preceding claim, further comprising isolating a compound of interest from the culture medium.

7. The method or use to any preceding claim, wherein the yield of the compound of interest is increased by at least about 100% when compared to when the host cell is cultured in the absence of peptone.

8. The method or use of any preceding claim, wherein the method further comprises a step of formulating the compound of interest into a pharmaceutical composition or an agrochemical composition.

9. The method or use of any preceding claim, wherein the filamentous fungal host cell is selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonii, Trichoderma reesei, Penicillium chrysogenum, Penicillium oxalicum and Neurospora crassa.

10. The method or use of claim 9, wherein the microbial host cell is a cell of Trichoderma reesei.

11. The method or use of any preceding claim, wherein the at least one polynucleotide coding for the compound of interest is operably linked to a promoter, optionally to an inducible promoter.

12. (canceled)

13. The method or use of any proceeding claim, wherein the antibody or functional fragment thereof of is a heavy chain variable domain of a heavy chain antibody or a functional fragment thereof (VHH).

14. The method or use of claim 13, wherein the VHH is a VHH comprising:
a. a CDR1 comprising or consisting of a sequence selected from the group consisting of SEQ ID NOs 3, 8 and 13;
b. a CDR2 comprising or consisting of a sequence selected from the group consisting of SEQ ID NOs: 4, 9 and 14; and
c. a CDR3 comprising or consisting of a sequence selected from the group consisting of SEQ ID NOs: 5, 10 and 15.

15. The method or use of claim 13, wherein the VHH is a VHH comprising:
d. a CDR1 comprising or consisting of the sequence of SEQ ID NO: 3, a CDR2 comprising or consisting of the sequence of SEQ ID NO: 4 and a CDR3 comprising or consisting of the sequence of SEQ ID NO: 5;
e. a CDR1 comprising or consisting of the sequence of SEQ ID NO: 8, a CDR2 comprising or consisting of the sequence of SEQ ID NO: 9 and a CDR3 comprising or consisting of the sequence of SEQ ID NO: 10 or
f. a CDR1 comprising or consisting of the sequence of SEQ ID NO: 13, a CDR2 comprising or consisting of the sequence of SEQ ID NO: 14 and a CDR3 comprising or consisting of the sequence of SEQ ID NO: 15.

16. The method or use of claim 13, wherein the VHH is a VHH comprising or consisting of a sequence selected from the group consisting of SEQ ID NOs: 1, 2, 6, 7, 11 and 12.

17. The method or use of claim 13, wherein the VHH is a VHH comprising or consisting of SEQ ID NO: 1.

18. The method or use of claim 13, wherein the VHH is a VHH comprising or consisting of SEQ ID NO: 2.

19. The method or use of any preceding claim, wherein the method comprises inserting the polynucleotide coding for a compound of interest into the microbial host cell prior to culturing the host cell.

20. Use of a microbial host cell for the production of a compound of interest, wherein the microbial host comprises at least one polynucleotide coding for the compound of interest, and wherein the use comprises culturing the microbial host cell under conditions conducive to the expression of the compound of interest, wherein the microbial host is cultured in the presence of peptone.
wherein the microbial host cell is a filamentous fungal host cell,
wherein the compound of interest is an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof (VHH), a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain, and
wherein the yield of the compound of interest is increased compared to when the microbial host cell is cultured in the absence of peptone.

21. The use of a microbial host cell of claim 20, wherein the method is the method of any one of claims 1 to 19.

22. The method or use of claim 4, wherein the peptone is free of animal derived products.


SEQUENCE LISTING (15 sequences)

SEQ ID NO: 1 - protein, 119 aa, Artificial Sequence
  VHH1 D
DVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 2 - protein, 119 aa, Artificial Sequence
  VHH1 Q
QVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 3 - protein, 10 aa, Artificial Sequence
  VHH1 CDR1
RSIFSINAMD

SEQ ID NO: 4 - protein, 9 aa, Artificial Sequence
  VHH1 CDR2
GITRGGTTK

SEQ ID NO: 5 - protein, 11 aa, Artificial Sequence
  VHH1 CDR3
LRGEQPWTRDY

SEQ ID NO: 6 - protein, 117 aa, Artificial Sequence
  VHH2 Q
QVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 7 - protein, 117 aa, Artificial Sequence
  VHH2 D
DVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 8 - protein, 10 aa, Artificial Sequence
  VHH2 CDR1
GTIFRPTAMG

SEQ ID NO: 9 - protein, 9 aa, Artificial Sequence
  VHH2 CDR2
TITTGGSTK

SEQ ID NO: 10 - protein, 9 aa, Artificial Sequence
  VHH2 CDR3
QWGVRTRDY

SEQ ID NO: 11 - protein, 126 aa, Artificial Sequence
  VHH3 Q
QVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 12 - protein, 126 aa, Artificial Sequence
  VHH3 D
DVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 13 - protein, 10 aa, Artificial Sequence
  VHH3 CDR1
ISDRAFSRHV

SEQ ID NO: 14 - protein, 10 aa, Artificial Sequence
  VHH3 CDR2
AIGWTGRRTY

SEQ ID NO: 15 - protein, 15 aa, Artificial Sequence
  VHH3 CDR3
SHFYSVSFEINDYDY